(19) United States
(12) Patent Application Publication
(10) Pub. No.: US 2026/0053475 A1
(43) Pub. Date: Feb. 26, 2026

(54) MEDICAL IMAGE PROCESSING DEVICE, ULTRASOUND DIAGNOSTIC DEVICE, MEDICAL IMAGE PROCESSING METHOD, AND STORAGE MEDIUM

(71) Applicant: CANON MEDICAL SYSTEMS CORPORATION, Otawara-shi (JP)

(72) Inventors: Mio UTSUNOMIYA, Nasushiobara (JP); Masaki WATANABE, Utsunomiya (JP); Kousuke NAMIKI, Otawara (JP); Hiroaki ISHIKAWA, Omiya (JP); Yu IGARASHI, Utsunomiya (JP)

(73) Assignee: CANON MEDICAL SYSTEMS CORPORATION, Otawara-shi (JP)

(21) Appl. No.: 19/303,851

(22) Filed: Aug. 19, 2025

(30) Foreign Application Priority Data
Aug. 21, 2024 (JP) 2024-139734

(51) Int. Cl. A61B 8/00

(52) U.S. Cl. CPC A61B 8/5207; A61B 8/463

(57) ABSTRACT

In a medical image processing device, a processing circuitry sequentially generates ultrasound still images associated with time information indicating an elapsed time until a reflected wave signal is collected from a reference time based on a reflected wave signal of an ultrasound signal transmitted toward a subject, generates an ultrasound moving image including the ultrasound still images for a period according to a predetermined image capturing situation for a purpose of an ultrasound examination, outputs identification information indicating the identified image capturing situation based on the time information associated with the ultrasound still images included in the ultrasound moving image, and causes a display device to display the ultrasound image corresponding to the identification information that has been selected and the time information in association with at least one of the ultrasound moving image and the ultrasound still images, the time information, and the identification information.

### CROSS-REFERENCE TO RELATED APPLICATION

The present application claims priority based on Japanese Patent Application No. 2024-139734, filed Aug. 21, 2024, the content of which is incorporated herein by reference.

### FIELD

Embodiments disclosed in the present specification and drawings relate to a medical image processing device, an ultrasound diagnostic device, a medical image processing method, and a storage medium.

### BACKGROUND

Conventionally, for example, in the differential diagnosis of hepatocellular carcinoma, an ultrasound contrast examination in which a contrast agent is injected into a subject (a patient) and an ultrasound diagnostic device captures an image of an examination target area is performed. In the ultrasound contrast examination, a doctor starts a timer when the contrast agent is injected into the subject and the ultrasound diagnostic device captures an image for a predetermined time. At this time, the ultrasound diagnostic device associates time information measured by the timer with ultrasound images (moving images) captured for a predetermined period of time at each preset (programmed) timing or at each timing according to a doctor's operation and stores association results. After an imaging process ends in the ultrasound contrast examination, the doctor ascertains the stored ultrasound images (moving images), specifies the ultrasound images (still images) to be attached to a report, and stores (restores) the specified ultrasound images in the ultrasound diagnostic device. Subsequently, the doctor inputs a diagnostic opinion for the subject on the basis of the restored ultrasound images (still images) and the time information associated with these ultrasound images, and creates a report to which the specified (restored) ultrasound images (still images) are attached.

However, the number of ultrasound images (moving images and still images) captured in an ultrasound contrast examination is significantly large. For this reason, the doctor's task of specifying the ultrasound images (still images) to be attached to the report from a large number of ultrasound images is troublesome.

In relation to this, technology related to an image diagnostic device for setting a plurality of group information items according to the time elapsed from a reference time, adding group information corresponding to the elapsed time, and storing diagnostic images and an image display device for displaying group-specific diagnostic images using the group information has been proposed. By employing this technology, it is considered that the examination time of an ultrasound contrast examination, particularly, the time required for a doctor to specify ultrasound images (still images), can be reduced.

Here, the time when an ultrasound image (a moving image or still image) is captured, i.e., the time elapsed after a contrast agent is injected into the subject, is also a determination factor when the subject is diagnosed. Even in conventional technology, when diagnostic opinions are input or reports are created, it is necessary to be careful not to erroneously link a specified ultrasound image (still image), time information associated with this ultrasound image, and the diagnostic opinion. For this reason, it is considered that conventional technology has not yet improved the efficiency of doctors' work that is performed during an ultrasound contrast examination.

### BRIEF DESCRIPTION OF THE DRAWINGS

FIG. 1 is a diagram showing an example of a functional configuration of a medical image processing device according to an embodiment and a functional configuration and usage environment of an ultrasound diagnostic device to which the medical image processing device according to the embodiment is applied.

FIG. 2 is a diagram showing an example of a display screen displayed on a display device by the medical image processing device according to the embodiment.

FIG. 3 is a diagram showing an example of an ultrasound image displayed in an image display area included in a display screen displayed on the display device by the medical image processing device according to the embodiment.

FIG. 4 is a diagram showing an example of time phase information displayed in a time phase display area included in a display screen displayed on the display device by the medical image processing device according to the embodiment.

FIG. 5 is a diagram showing an example of time information displayed in a time axis area included in a display screen displayed on the display device by the medical image processing device according to the embodiment.

FIG. 6 is a diagram schematically showing an example of a state in which the medical image processing device according to the embodiment provides information displayed on the display device.

FIG. 7 is a flowchart showing an example of a flow of a process in the medical image processing device according to the embodiment.

### DETAILED DESCRIPTION

According to an embodiment, there is provided a medical image processing device includes processing circuitry. The processing circuitry sequentially generates ultrasound still images associated with time information indicating an elapsed time until a reflected wave signal is collected from a reference time on the basis of a reflected wave signal when an ultrasound signal transmitted toward a subject is reflected inside a body of the subject; generates an ultrasound moving image including the ultrasound still images for a period according to a predetermined image capturing situation for a purpose of an ultrasound examination; identifies the image capturing situation on the basis of the time information associated with the ultrasound still images included in the ultrasound moving image; outputs identification information indicating the identified image capturing situation; and causes a display device to display an ultrasound image of at least one of the ultrasound moving image and the ultrasound still images, the time information, and the identification information in association with one another. The processing circuitry causes the display device to display the ultrasound image corresponding to the identification information that has been selected and the time information.

Hereinafter, a medical image processing device, an ultrasound diagnostic device, a medical image processing method, and a storage medium of embodiment will be described with reference to the drawings. The medical image processing device presents an ultrasound image captured by the ultrasound diagnostic device and various types of information related to the ultrasound image. In the following description, a case where the medical image processing device is applied to the ultrasound diagnostic device of an embodiment will be described as an example. The ultrasound diagnostic device, for example, performs an ultrasound examination on a subject by transmitting an ultrasound signal from an ultrasound probe and detecting an ultrasound signal (a reflected wave signal: echo signal) that is the transmitted ultrasound signal reflected inside the body of the subject (patient) and returns with the ultrasound probe. The ultrasound diagnostic device generates an ultrasound image based on a magnitude of the reflected wave signal or the like by performing image processing on the reflected wave signal detected by the ultrasound probe and presents the generated ultrasound image to an examiner (such as a doctor or technician) performing the ultrasound examination by causing a display device to display the ultrasound image. Thereby, the examiner can visually ascertain a state of the tissue inside the subject's body. When the ultrasound image generated by the ultrasound diagnostic device is presented to the examiner, the medical image processing device presents various types of information related to the ultrasound image so that the examiner can easily ascertain the ultrasound image and perform the ultrasound examination more suitably.

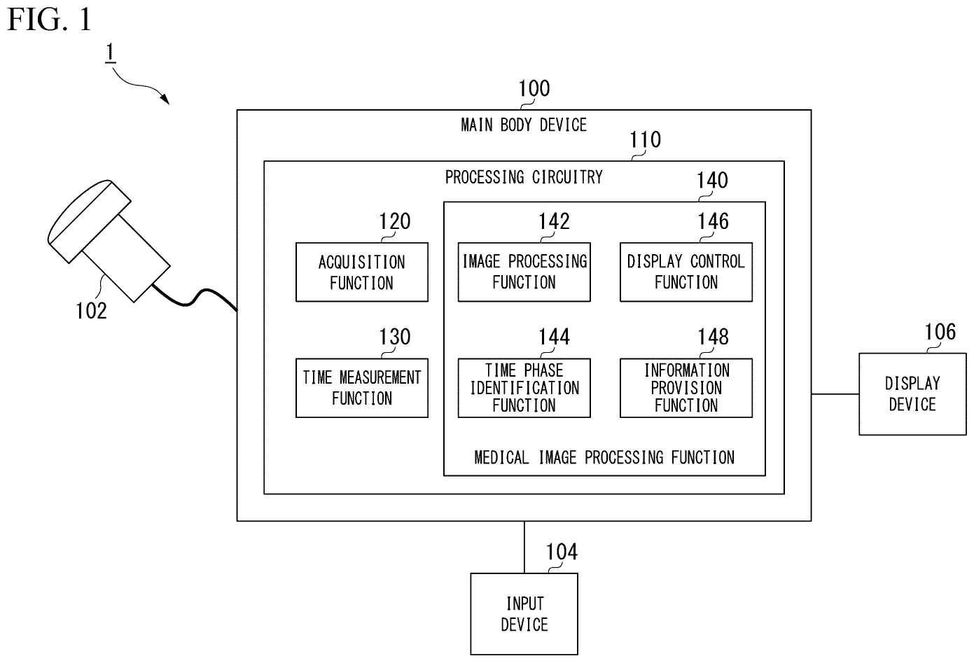

FIG. 1 is a diagram showing an example of a functional configuration of a medical image processing device according to an embodiment and a functional configuration and usage environment of an ultrasound diagnostic device to which the medical image processing device according to the embodiment is applied. The ultrasound diagnostic device 1 includes, for example, a main body device 100, an ultrasound probe 102, an input device 104, and a display device 106. Although the input device 104 and the display device 106 are connected to the main body device 100 in FIG. 1, a configuration in which the input device 104 and the display device 106 are incorporated in the main body device 100 may be adopted.

In FIG. 1, for example, an example of the functional configuration and usage environment of the ultrasound diagnostic device 1 used to perform an ultrasound contrast examination in which a contrast agent is injected into a subject and an image of an examination target area is captured to perform a differential diagnosis of hepatocellular carcinoma is shown. On the other hand, the illustration of other constituent elements and functional configurations that implements functions not related to the ultrasound contrast examination on the subject in the ultrasound diagnostic device 1 is omitted in FIG. 1. For example, the illustration of an input/output circuit configured to output a control signal for transmitting an ultrasound signal to the ultrasound probe 102 and receive a reflected wave signal output by the ultrasound probe 102 and a control circuit (a control function) configured to control the whole of the ultrasound diagnostic device 1 and the main body device 100 is omitted in FIG. 1. It is only necessary for the configurations and operations of these omitted constituent elements and functional configurations to be equivalent to the configurations and operations of the constituent elements and functional configurations of existing ultrasound diagnostic devices. Therefore, detailed descriptions of the configurations and operations of the omitted constituent elements and functional configurations will be omitted.

The ultrasound probe 102 is used in a state in which the ultrasound probe 102 abuts against or is close to a surface of the subject's body. The ultrasound probe 102 transmits an ultrasound signal having directionality to the subject's body in accordance with control from the main body device 100, detects a reflected wave signal reflected inside the subject's body, and outputs the reflected wave signal to the main body device 100. The ultrasound probe 102 has a plurality of ultrasonic transducers. The ultrasonic transducers are, for example, piezoelectric elements such as piezoelectric ceramics. The ultrasound probe 102 further includes a matching layer provided in each of the ultrasonic transducers, a backing material that prevents the propagation of the ultrasound signal to the rear of the ultrasonic transducers (the opposite side of the subject), and the like. A plurality of ultrasonic transducers are arrayed inside the ultrasound probe 102 in any array method, such as a single row or a two-dimensional array. The ultrasound probe 102 may be detachable from the main body device 100.

The ultrasound probe 102 and the main body device 100 may be connected by a dedicated cable or by a wireless communication function.

The input device 104 receives an operation on the ultrasound diagnostic device 1 (more specifically, the main body device 100) by the examiner, and outputs information (an electrical signal) indicating the received operation to the main body device 100. The input device 104 is implemented by, for example, a mouse, a keyboard, a touch panel, a microphone, and the like. When the input device 104 is a touch panel, the input device 104 may be formed integrally with a display device such as a terminal device, a personal computer (PC), or a display device such as the display device 106 connected to the main body device 100. The input device 104 may be implemented by a terminal device, a personal computer, or a display device (e.g., a tablet terminal) capable of wireless communication with the main body device 100. In the present specification, the input device 104 is not limited to a device having physical operation parts such as the mouse and keyboard described above. For example, an electrical signal processing circuit for receiving an electrical signal corresponding to an input operation from a terminal device, a personal computer, or an external input device provided separately from the main body device 100 and outputting this electrical signal to the terminal device, the personal computer, or the main body device 100 is also included in the example of the input device 104.

The display device 106 is, for example, a liquid crystal display (LCD), a cathode ray tube (CRT) display, an organic electroluminescence (EL) display, or the like. The display device 106 displays various types of information including an ultrasound image to be presented to the examiner. Thereby, the examiner can visually ascertain the state of the tissues inside the subject's body by looking at the ultrasound image displayed on the display device 106. The various types of information displayed by the display device 106 also include information about a graphical user interface (GUI) (a GUI image) for receiving various types of input operations (e.g., an ultrasound image acquisition start instruction) from the examiner to the ultrasound diagnostic device 1, i.e., the input device 104, and the like.

The main body device 100 causes the ultrasound probe 102 to transmit an ultrasound signal and generate an ultrasound image (an echo image) that is a still picture on the basis of the reflected wave signal detected and output by the ultrasound probe 102. Furthermore, the main body device 100 generates an ultrasound moving image based on the generated still ultrasound image (hereinafter referred to as “ultrasound still image”). The main body device 100 presents the generated ultrasound still image or ultrasound moving image to the examiner by outputting the images to the display device 106 so that the display device 106 is allowed to display the image.

### [Configuration of Main Body Device]

The main body device 100 includes, for example, processing circuitry 110.

The processing circuitry 110 executes processes of an acquisition function 120, a time measurement function 130, a medical image processing function 140, and the like. Processes of an image processing function 142, a time phase identification function 144, a display control function 146, an information provision function 148, and the like are executed by the medical image processing function 140.

The processing circuitry 110 implements the functions of the acquisition function 120, the time measurement function 130, and the medical image processing function 140 (including the image processing function 142, the time phase identification function 144, the display control function 146, and the information provision function 148), for example, when a hardware processor executes a program (software) stored in a memory (a storage) (not shown). The memory (not shown) is implemented by, for example, a read-only memory (ROM), a random-access memory (RAM), a semiconductor memory element such as a flash memory, a hard disk drive (HDD), an optical disc, or the like.

The hardware processor is, for example, circuitry such as a central processing unit (CPU), a graphics processing unit (GPU), a large-scale integration (LSI) circuit, a system on chip (SOC), an application-specific integrated circuit (ASIC), or a programmable logic device (for example, a simple programmable logic device (SPLD), a complex programmable logic device (CPLD), or a field programmable gate array (FPGA)). Instead of storing the program in the memory (not shown), the program may be directly embedded in the circuitry of the hardware processor. In this case, the hardware processor implements each function by reading and executing the program embedded in the circuitry. The hardware processor is not limited to being configured as single circuitry and may be configured as one hardware processor by combining a plurality of independent circuits to implement each function. A plurality of constituent elements may be integrated into one hardware processor to implement each function. Each function may be implemented by incorporating a plurality of constituent elements into one dedicated LSI circuit. Here, the program (software) may be stored in advance in a storage device (a storage device having a non-transitory storage medium) that constitutes a semiconductor memory device such as a ROM, a RAM, or a flash memory or a storage device such as a hard disk drive. Alternatively, the program (software) may be stored in a removable storage medium (a non-transitory storage medium) such as a DVD or CD-ROM and installed in a storage device (not shown) provided in the main body device 100 when the storage medium is mounted in a drive device provided in the main body device 100. The program (software) may be downloaded in advance from another computer device via a network (not shown) and installed in the storage device provided in the main body device 100. A program (software) installed in the storage device provided in the main body device 100 may be transferred to the processing circuitry provided in the main body device 100 and executed.

The acquisition function 120 is executed to acquire (collect) the reflected wave signal output by the ultrasound probe 102. The acquisition function 120 is executed to output the acquired reflected wave signal to the medical image processing function 140. The acquisition function 120 is an example of an “acquirer.”

The time measurement function 130 is, for example, a timer. The time measurement function 130 is activated and starts the time measurement if the examiner performs an input operation for issuing a time measurement start instruction to the input device 104. The examiner instructs the time measurement function 130 to start the time measurement at a reference timing (hereinafter referred to as a “reference time”) in the ultrasound contrast examination. The reference time is, for example, a timing when a contrast agent is injected into the subject to perform the ultrasound contrast examination. Therefore, the time measured by the time measurement function 130 is the elapsed time until the acquisition function 120 is executed to acquire (collect) the reflected wave signal from the start of the ultrasound contrast examination. The time measurement function 130 is executed to output time information indicating the measured time to the medical image processing function 140.

The time measurement function 130 is an example of a “time measurementer.”

The medical image processing function 140 is executed to generate ultrasound still images and ultrasound moving images on the basis of the reflected wave signals output by the acquisition function 120 and output the generated ultrasound still images and ultrasound moving images to the display device 106 so that the display device 106 is allowed to display the images.

The medical image processing function 140 is an example of a “medical image processing device.”

The image processing function 142 is executed to sequentially generate ultrasound still images on the basis of the reflected wave signals output by the acquisition function 120. At this time, the image processing function 142 is executed to associate the time information output by the time measurement function 130 with each generated ultrasound still image. The image processing function 142 sequentially generates ultrasound moving images obtained by combining ultrasound still images for a predetermined period. A period for which the ultrasound still images are combined (in other words, the period for which the ultrasound moving images are captured) is a period according to an image capturing situation that is predetermined for the purpose of the ultrasound examination. The period for which the ultrasound still images are combined, for example, is set in advance by a setting function (not shown) when a combination of times, taking clinical significance into account, is programmed in advance and the program is read and executed by a setting function (not shown). The period for which the ultrasound still images are combined is, for example, a period corresponding to a time phase that is predetermined in the guideline for the ultrasound contrast examination or a period designated by the examiner. The time phase indicates a situation of an examination target area determined by the elapsed time from a reference time (when the contrast agent is injected into the subject). The period for which the ultrasound still images are combined may be, for example, a period corresponding to one heartbeat of the subject. For example, the start and end times of the ultrasound moving images are set as the period for which the ultrasound still images are combined. The image processing function 142 is executed to sequentially generate ultrasound moving images, each of which is a combination of ultrasound still images between the set start and end times. The image processing function 142 is executed to sequentially output the generated ultrasound moving images to the time phase identification function 144 and the display control function 146.

Meanwhile, in an ultrasound contrast examination, after a process for capturing a series of images ends in the ultrasound contrast examination, the examiner ascertains the ultrasound moving images and specifies an ultrasound still image to be attached when a diagnostic opinion is input or a report is created. In other words, an ultrasound still image of one frame constituting the ultrasound moving image is specified. In this case, the image processing function 142 is executed to extract (pull out) the specified ultrasound still image from among a plurality of ultrasound still images included in the period in which they are combined as the ultrasound moving image and generate it as another ultrasound still image (a new ultrasound still image). The image processing function 142 is executed to output the specified ultrasound still image again to the time phase identification function 144 and the display control function 146.

The image processing function 142 may be configured to cause a storage (not shown) included in the main body device 100 to sequentially store the ultrasound moving images and the ultrasound still images that have been generated and output a notification indicating this storage to each of the time phase identification function 144 and the display control function 146. This case, for example, is equivalent to a configuration in which the time phase identification function 144 is executed to read the ultrasound moving images and the ultrasound still images stored in the storage (not shown) and therefore the image processing function 142 is executed to output the ultrasound moving images and the ultrasound still images that have been generated to the time phase identification function 144.

The image processing function 142 may be executed to transmit the ultrasound moving images and the ultrasound still images that have been generated to a storage device (not shown) connected via a network (not shown) such as a local area network (LAN) constructed in a hospital so that the images are stored in the storage device. The storage device (not shown) is, for example, a storage device provided in a database system such as a medical image management system (a picture archiving and communication system (PACS) that manages data such as various types of medical images and ultrasound images captured in ultrasound examinations (including ultrasound contrast examinations) previously performed on the subject.

The image processing function 142 is an example of an “image processor.”

The time phase is an example of an “image capturing situation.”

The time phase identification function 144 is executed to identify a time phase in an ultrasound contrast examination on the basis of time information associated with ultrasound still images combined into each of the ultrasound moving images sequentially output by the image processing function 142 and time information associated with an ultrasound still image output again by the image processing function 142. More specifically, a time phase corresponding to the ultrasound moving image or the ultrasound still image in the ultrasound contrast examination is identified. When the ultrasound moving images or ultrasound still images are stored in the storage device (not shown) included in the database system such as the medical image management system, the time phase identification function 144 may be executed to read the target ultrasound moving images or ultrasound still images from the storage device (not shown) and identify the time phases of the ultrasound moving images or ultrasound still images. The time phase identification function 144 is executed to sequentially output information such as a time phase name indicating the identified time phase (hereinafter referred to as “time phase information”) to the display control function 146. At this time, the time phase identification function 144 is executed to output the time phase information and information indicating the ultrasound moving images or ultrasound still images for which the time phases have been identified in association with each other to the display control function 146. Furthermore, when the time phase of the ultrasound still image is identified, the time phase identification function 144 is executed to output information indicating the identification from the ultrasound still image in association with the time phase information to the display control function 146.

The time phase identification function 144 may be executed to sequentially output the time phase information in association with the ultrasound moving images or the ultrasound still images, i.e., sequentially output the ultrasound moving images or the ultrasound still images with which the time information and the time phase information are associated, to the display control function 146. In this case, the output of the ultrasound moving images or the ultrasound still images from the image processing function 142 to the display control function 146 may be omitted. The time phase identification function 144 may be configured to cause a storage (not shown) provided in the main body device 100 to sequentially store the ultrasound moving images or ultrasound still images with which time phase information is associated, and output a notification indicating this storage to the display control function 146. This case is equivalent to a configuration in which the display control function 146 is executed to read the ultrasound moving images or ultrasound still images stored in the storage (not shown) and therefore the time phase identification function 144 is executed to output the ultrasound moving images or ultrasound still images with which the time phase information is associated to the display control function 146.

The time phase identification function 144 is an example of an “identifier.”

The time phase information (including a time phase name) is an example of “identification information.”

The display control function 146 is executed to present information obtained by associating the ultrasound moving images and ultrasound still images (including time information) output sequentially by the image processing function 142 with the time phase information output sequentially by the time phase identification function 144 as image information to the examiner. More specifically, the display control function 146 is executed to generate display images (display screens) including the ultrasound moving images and ultrasound still images (including the time information) output by the image processing function 142 and the time phase information output by the time phase identification function 144, sequentially output the generated display images to the display device 106 so that the generated display images are displayed, and present the display images to the examiner. At this time, the display control function 146 may be executed to superimpose a GUI image or the like on the display image and display the superimposed image on the display device 106.

The GUI image includes, for example, an operation button to be operated when an ultrasound still image to be attached during a process for inputting the diagnostic opinion or creating a report is specified by the examiner ascertaining an ultrasound moving image after a process for capturing a series of images in an ultrasound contrast examination ends, an activation button for activating another application linked to an application or the like for use when the diagnostic opinion is input or the report is created, and the like. It is only necessary for these buttons to be displayed (arranged) at suitable positions within the display screen.

The operation buttons are, for example, buttons for playing, stopping, pausing, fast-forwarding, rewinding, and storing (restoring ultrasound still images) the ultrasound moving image ascertained by the examiner and the like. By operating the operation buttons, the examiner can specify (select) an ultrasound still image of one frame constituting the ultrasound moving image, and store (restore) the specified (selected) ultrasound still image in the main body device 100. At this time, the display control function 146 is executed to output information indicating the ultrasound still image of one frame specified (selected) by the examiner to the image processing function 142. Thereby, the image processing function 142 is executed to generate the ultrasound still image indicated in the information output by the display control function 146 as a new ultrasound still image and output the ultrasound still image again to each of the time phase identification function 144 and the display control function 146.

The activation button is, for example, a button for activating other applications prepared to improve the efficiency of the examiner's work in ultrasound contrast examinations, such as an opinion input application used when diagnostic opinion are input, a report creation application used when a report according to a guideline for the ultrasound contrast examination or the like is created on the basis of a diagnostic opinion, and an analysis application for analyzing ultrasound moving images and ultrasound still images to obtain diagnostic results. The examiner can shorten the time required to obtain the results of the ultrasound contrast examination by operating the activation button to activate a necessary application and associating various types of information including the ultrasound still image obtained by the medical image processing function 140 (the medical image processing device) on the activated application.

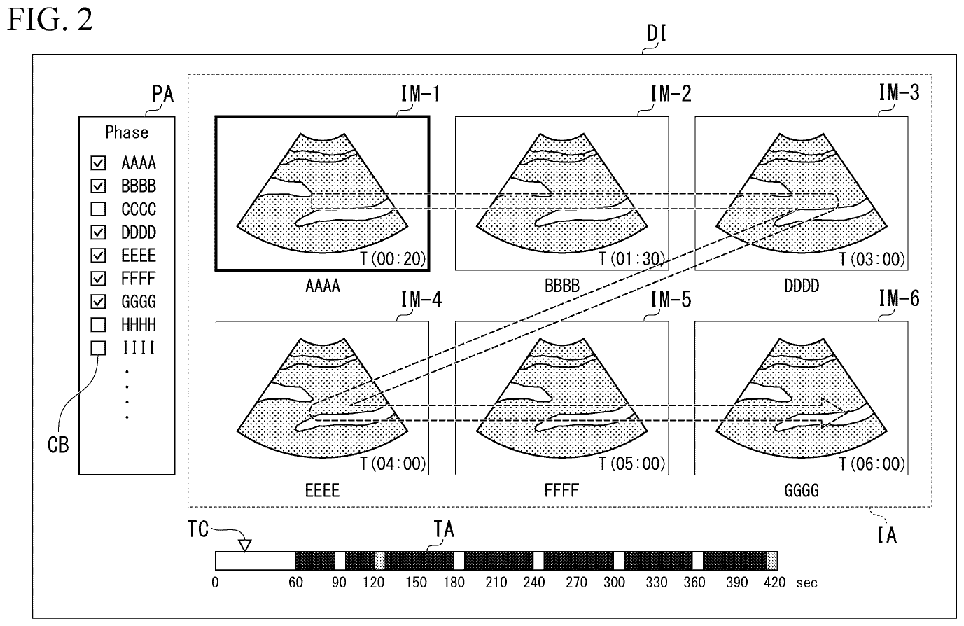

The display control function 146 is an example of a “display controller.” Here, an example of a display image (a display screen) generated by the display control function 146 and displayed on the display device 106 will be described. FIG. 2 is a diagram showing an example of a display screen displayed on the display device 106 by the medical image processing device (the medical image processing function 140) according to the embodiment. A display screen DI shown in FIG. 2 includes an image display area IA, a time phase display area PA, and a time axis area TA.

The image display area IA is an area for displaying a predetermined number of ultrasound moving images or ultrasound still images among the plurality of ultrasound images (ultrasound moving images and/or ultrasound still images) output by the image processing function 142 in a series of ultrasound contrast examinations. The display screen DI shown in FIG. 2 shows an example in which six ultrasound moving images IM (ultrasound moving images IM-1 to IM-6) are arranged in the image display area IA and displayed as one screen. As the ultrasound moving image IM, the ultrasound still image of the first or last frame constituting the ultrasound moving image is displayed. The number of ultrasound moving images or ultrasound still images displayed (arranged) within the image display area IA is not limited to six, and may be less than six (e.g., four) or more than six (e.g., nine). When the number of ultrasound moving images or ultrasound still images displayed (arranged) in the image display area IA is larger, it may be switched, for example, by a scroll bar, page movement, or the like. The ultrasound moving images or the ultrasound still images to be displayed on the display screen DI are selected (designated) in the time phase display area PA. The display control function 146 is executed to arrange each ultrasound image (ultrasound moving image and/or ultrasound still image) selected (designated) in the time phase display area PA in chronological order and arrange the images in the image display area IA. In the display screen DI shown in FIG. 2, an example of the case where the ultrasound moving images IM-1 to IM-3 are arranged in chronological order from the top left to the top right in the image display area IA and then the ultrasound moving images IM-4 to IM-6 are arranged in chronological order from the bottom left to the bottom right through a process for returning to the bottom left of the image display area IA, in other words, the ultrasound moving images are arranged in raster order, is shown.

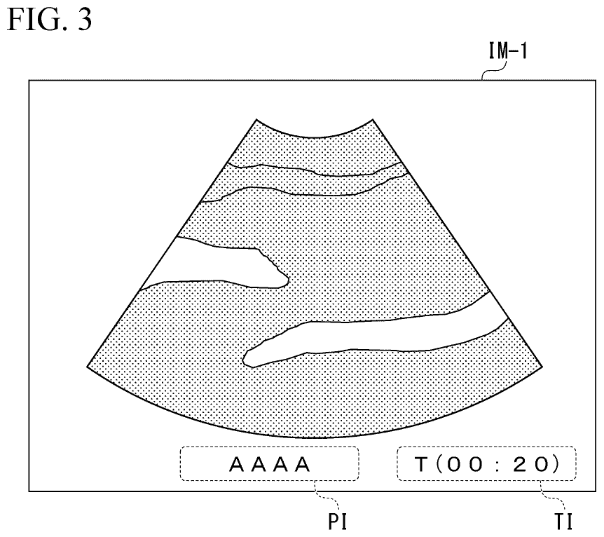

Time information and time phase information that are associated are superimposed on each ultrasound moving image or ultrasound still image displayed in the image display area IA. FIG. 3 is a diagram showing an example of an ultrasound image (an ultrasound moving image IM) displayed in the image display area IA included in the display screen DI displayed on the display device 106 by the medical image processing device (the medical image processing function 140) according to the embodiment. In FIG. 3, the time phase name “AAAA” is displayed as time phase information PI within the image of the ultrasound moving image IM-1 and the elapsed time (20 sec) from the reference time (when the contrast agent was injected into the subject) associated with the currently displayed frame (i.e., ultrasound still image) of the ultrasound moving image IM-1 is displayed as time information TI. Although an example in which both the time phase information PI and the time information TI are superimposed on the image of the ultrasound moving image IM-1 is shown in FIG. 3, a position at which the time phase information PI and the time information TI are superimposed may be any positions as long as it can be visually ascertained that the time phase information PI and the time information TI correspond to the ultrasound moving image or the ultrasound still image. For example, as in the display screen DI shown in FIG. 2, only the time information TI may be superimposed inside the image of the ultrasound moving image IM, and the time phase information PI may be superimposed outside the image of the ultrasound moving image IM. That is, either or both of the time phase information PI and the time information TI may be superimposed inside the image that is the ultrasound moving image or ultrasound still image or may be superimposed outside the image that is the ultrasound moving image or ultrasound still image.

The image display area IA is an example of an “image display area.”

The time phase display area PA is an area for displaying the time phase information output by the time phase identification function 144 in a series of ultrasound contrast examinations. In the time phase display area PA, the time phase names included in the time phase information identified by the time phase identification function 144 from ultrasound moving images or ultrasound still images are displayed in chronological order. Furthermore, a GUI image for selecting (designating) the ultrasound moving image to be displayed in the image display area IA is superimposed in the time phase display area PA. The display screen DI shown in FIG. 2 shows an example in which the time phase names of the ultrasound moving images IM are displayed in the time phase display area PA in chronological order, and check boxes CB corresponding to the time phase names are displayed. When the examiner performs an input operation for selecting (designating) a time phase name using the input device 104, the display control function 146 is executed to arrange the selected (designated) ultrasound moving images IM in chronological order in the image display area IA. In the display screen DI shown in FIG. 2, an example of a case where an ultrasound moving image of a time phase name is selected (designated) by checking a check box CB corresponding to the time phase name is shown. Thereby, the display control function 146 is executed to display (arrange) the ultrasound moving images corresponding to time phase names each having a check mark in the check box CB in the time phase display area PA as selected (designated) ultrasound moving images or ultrasound still images in the image display area IA of the display screen DI. The display screen DI shown in FIG. 2 shows an example in which an ultrasound moving image IM-1 corresponding to a time phase name “AAAA,” an ultrasound moving image IM-2 corresponding to a time phase name “BBBB,” an ultrasound moving image IM-3 corresponding to a time phase name “DDDD,” an ultrasound moving image IM-4 corresponding to a time phase name “EEEE,” an ultrasound moving image IM-5 corresponding to a time phase name “FFFF,” and an ultrasound moving image IM-6 corresponding to a time phase name “GGGG” are arranged in the image display area IA.

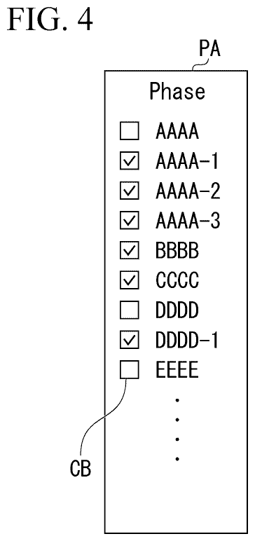

As described above, when the time phase identification function 144 is executed to identify the time phase of an ultrasound still image, information indicating the identification from the ultrasound still image is associated with the time phase information. Therefore, in the time phase display area PA, the time phase names included in the time phase information are displayed in chronological order to distinguish between the ultrasound moving image and the ultrasound still image of the same time phase, i.e., to distinguish between the original moving image and the still image restored from the original moving image. In FIG. 4, a diagram showing an example of time phase information (phase names) displayed in the time phase display area PA included in the display screen DI displayed on the display device 106 by the medical image processing device (the medical image processing function 140) according to the embodiment is shown. In FIG. 4, an example of the time phase display area PA in a case where an ultrasound still image is specified in each ultrasound moving image IM of an ultrasound moving image IM-1 corresponding to a time phase name “AAAA” and an ultrasound moving image IM-3 corresponding to a time phase name “DDDD” is shown. In FIG. 4, a “- (hyphen)” and a number are shown after the time phase name of the ultrasound still image specified from each ultrasound moving image IM. In other words, a sub-number is added to the time phase name. The sub-number, for example, may indicate a sequential order specified from the ultrasound moving image IM, indicate a time order within the ultrasound moving image IM, or indicate a frame number of the ultrasound still image within the ultrasound moving image IM. The sub-number added to the time phase name allows the examiner to distinguish between the ultrasound moving image IM of the same time phase and the ultrasound still image specified from the ultrasound moving image IM. Information for distinguishing between the ultrasound moving image IM of the same time phase and the ultrasound still image specified from the ultrasound moving image IM is not limited to the sub-number. For example, any information may be used as long as it can distinguish between the ultrasound moving image IM of the same time phase and the ultrasound still image specified from the ultrasound moving image IM, such as time information of the ultrasound still image inside the ultrasound moving image IM. When the ultrasound still image is specified from the ultrasound moving image IM, the display control function 146 is executed to replace the ultrasound moving image IM arranged inside the image display area IA with the specified ultrasound still image.

The time phase display area PA is an example of an “identification information area.” The check box CB is an example of an “identification information selection area.”

The time axis area TA is an area in which a single time axis represents the entire examination time from the reference time in the ultrasound contrast examination and time information associated with the ultrasound moving image and the ultrasound still image is indicated within this time axis. In the time axis area TA, information indicating a timing at which the ultrasound moving image or the ultrasound still image displayed in the image display area IA was captured in the ultrasound contrast examination is displayed. In other words, in the time axis area TA, segments or timings in which ultrasound moving images and ultrasound still images were generated (or captured) during a series of ultrasound contrast examinations and segment or timings in which ultrasound moving images and ultrasound still images were not generated (or not captured) is configured to be able to be visually ascertained. Also, it is possible to visually ascertain whether or not a corresponding ultrasound moving image or ultrasound still image has been displayed in the image display area IA in segments or timings in which the ultrasound moving images and the ultrasound still images were generated (or captured) in the time axis area TA. The display screen DI shown in FIG. 2 shows an example of the time axis area TA when the examination time of the ultrasound contrast examination is 420 [sec]. The scale of the time axis in the time axis area TA may not be information about time (here, in units of seconds) as shown in the display screen DI. For example, the time phase name may be displayed at the corresponding position as the scale of the time axis in the time axis area TA. The display control function 146 is configured to visually ascertain each segment or timing shown within the time axis area TA by changing the display aspect (for example, by changing a color, pattern (so-called hatching), or the like). Furthermore, in the time axis area TA, a mark indicating the time at which the ultrasound moving image or ultrasound still image currently selected within the time phase display area PA is displayed is displayed. The display screen DI shown in FIG. 2 shows a display time mark TC corresponding to the ultrasound moving image IM-1 selected within the time phase display area PA. When the examiner selects the ultrasound moving image IM-1, which is a moving image, the display control function 146 is executed to play the ultrasound moving image IM-1. That is, the display control function 146 is executed to sequentially display each frame (ultrasound still image) constituting the ultrasound moving image in chronological order. Also, the display control function 146 is executed to sequentially move the position of the display time mark TC to the position corresponding to the currently displayed frame. When playback of the ultrasound moving image IM-1 has ended, the display control function 146 is executed to repeat playback from the first frame of the ultrasound moving image IM-1. The display control function 146 may be executed to superimpose a GUI image of operation buttons (operation buttons such as play, stop, pause, fast forward, rewind, and store buttons (a restore button for ultrasound still images)) on the display screen DI to receive an operation of the examiner to play the ultrasound moving image IM-1.

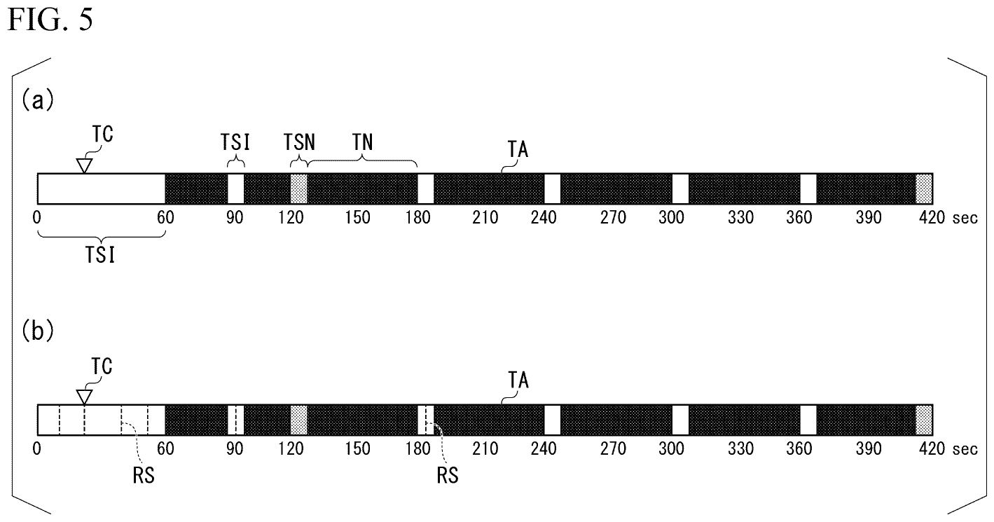

FIG. 5 is a diagram showing an example of time information displayed in a time axis area TA included in the display screen DI displayed on the display device 106 by the medical image processing device (the medical image processing function 140) according to the embodiment. In FIG. 5 (a), a period (a segment or timing) in which the ultrasound moving image or ultrasound still image displayed in the image display area IA was captured is represented by a captured image display period TSI, a captured image non-display period TSN, and a non-captured period TN. The captured image display period TSI is the period during which the ultrasound moving image displayed in the image display area IA was captured after being captured (stored). The captured image non-display period TSN is a period during which the ultrasound moving image was captured (stored) but not displayed in the image display area IA. The non-captured period TN is a period during which the ultrasound moving image was not captured (stored) and therefore could not be displayed in the image display area IA. In FIG. 5 (b), a still image restore mark RS indicates a timing at which the ultrasound still image (frame) specified in each ultrasound moving image was captured. In FIG. 5 (b), timings at which four ultrasound still images specified from ultrasound moving images captured during a time period of 0 to 60 sec were captured, a timing at which one ultrasound still image specified from ultrasound moving images captured during a time period of 90 to 95 sec was captured, and a timing at which one ultrasound still image specified from ultrasound moving images captured during a time period of 180 to 185 sec was captured are indicated by still image restore marks RS. When the display control function 146 is executed to replace an ultrasound moving image IM arranged within the image display area IA with an ultrasound still image corresponding to the still image restore mark RS, the display control function 146 is executed to indicate that the ultrasound moving image IM has been replaced with the ultrasound still image by changing a segment in the time axis area TA corresponding to the ultrasound moving image IM from a captured image display period TSI to a captured image non-display period TSN (changes the display aspect).

The time axis area TA is an example of a “time axis area.”

Returning to FIG. 1, the information provision function 148 is executed to provide various types of information related to the ultrasound contrast examination, such as the ultrasound still image included in the display screen DI displayed by the display control function 146 on the display device 106, and the time information and time phase information associated with this ultrasound still image. The examiner uses the input device 104 to issue an instruction to provide various types of information related to the ultrasound contrast examination (hereinafter referred to as “examination information”). For example, the examiner issues an instruction to provide examination information by operating an activation button of a GUI image to activate an opinion input application, a report creation application, or the like. The information provision function 148 is executed to output the examination information to an information receiver, such as the activated (indicated) opinion input application or report creation application.

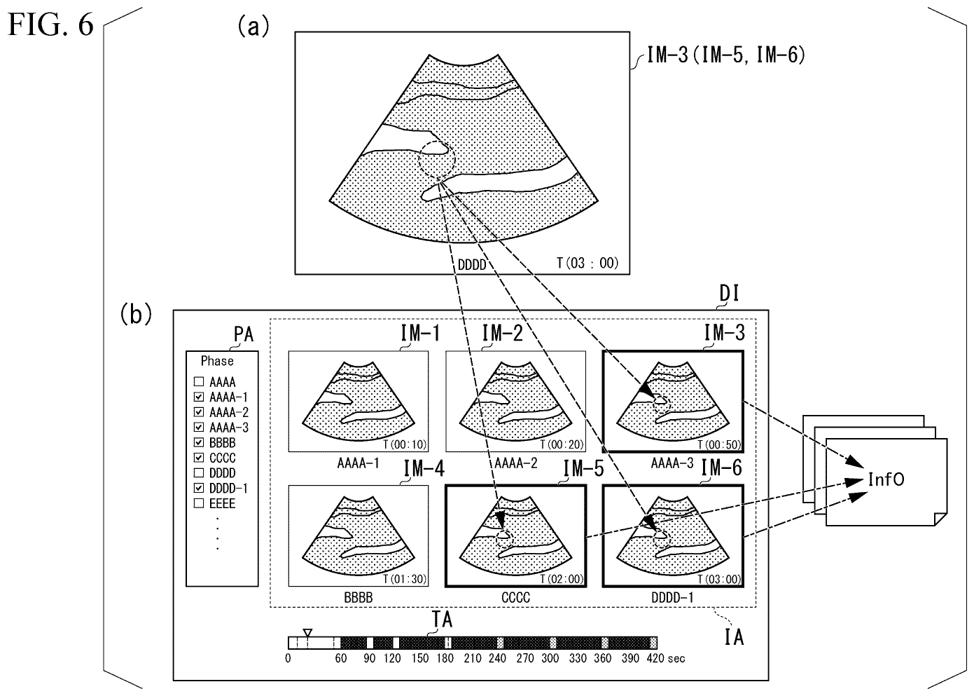

FIG. 6 is a diagram schematically showing an example of a state in which the medical image processing device (the medical image processing function 140) according to the embodiment provides information (examination information) displayed on the display device 106. In FIG. 6, for example, it is assumed that an area where there is concern the subject is afflicted is recognized within the ultrasound moving image IM-3, the ultrasound moving image IM-5, and the ultrasound moving image IM-6 displayed on the display screen DI by the analysis application. In FIG. 6 (a), a state in which an example of an area recognized by the analysis application is shown and a state in which the area is reflected in the display screen DI shown in FIG. 6 (b) is schematically shown. In this case, the examiner, for example, starts an opinion input application and inputs a diagnostic opinion for each ultrasound moving image IM. At this time, the information provision function 148 is executed to output examination information corresponding to each ultrasound moving image IM in which a diagnostic opinion have been input on the opinion input application to the opinion input application. In FIG. 6 (b), a state in which the ultrasound moving image IM-3, the ultrasound moving image IM-5, and the ultrasound moving image IM-6 to which a diagnostic opinion have been input and information such as time information and time phase information associated with these ultrasound moving images IM are output as output information InfO (provided to other applications linked to the opinion input application and the like) is schematically shown.

The information provision function 148 is an example of a “provisioner.”

### [Process of Ultrasound Diagnostic Device]

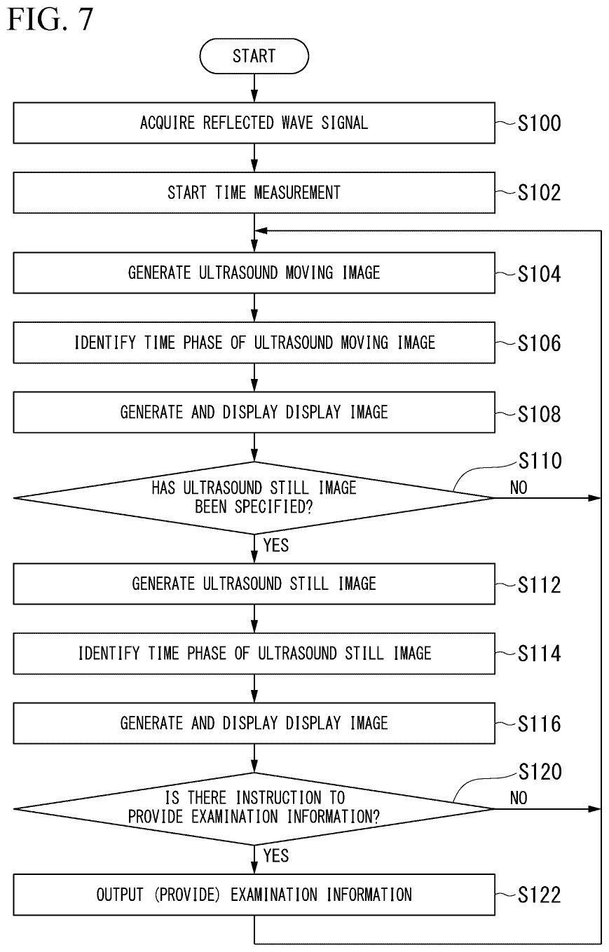

Next, an operation for displaying the display screen DI including the captured ultrasound moving image and ultrasound still image on the display device 106 and providing the examination information displayed on the display screen DI in the ultrasound diagnostic device 1 will be described. FIG. 7 is a flowchart showing an example of a flow of a process of the medical image processing device (the medical image processing function 140) according to the embodiment. The process of this flowchart is executed when the examiner performs an ultrasound contrast examination. In the following description, it is assumed that information indicating a timing at which the ultrasound moving image is generated and a length (period) of the generated ultrasound moving image is set (programmed) in advance in a setting function (not shown).

When the examiner starts an ultrasound contrast examination using the ultrasound diagnostic device 1, the acquisition function 120 is executed to acquire a reflected wave signal output by the ultrasound probe 102 (step S100). The acquisition function 120 is executed to output the acquired reflected wave signal to the medical image processing function 140. Subsequently, when the examiner issues an instruction to start time measurement according to an input operation of the input device 104, the time measurement function 130 is executed to start time measurement (step S102). The time measurement function 130 is executed to output measured time information to the medical image processing function 140. Subsequently, the acquisition function 120 is executed to repeat the acquisition of the reflected wave signal output by the ultrasound probe 102 and the output of the acquired reflected wave signal to the medical image processing function 140, and the time measurement function 130 is executed to repeat the output of the measured time information to the medical image processing function 140.

The image processing function 142 provided in the medical image processing function 140 is executed to sequentially generate ultrasound still images on the basis of the reflected wave signal output by the acquisition function 120 and generate an ultrasound moving image obtained by combining ultrasound still images for a preset (programmed) period (step S104). The image processing function 142 is executed to output the generated ultrasound moving image to each of the time phase identification function 144 and the display control function 146.

The time phase identification function 144 is executed to identify a time phase of the ultrasound moving image on the basis of the time information associated with the ultrasound moving images sequentially output by the image processing function 142 (step S106). The time phase identification function 144 is executed to sequentially output the identified time phase information to the display control function 146.

The display control function 146 is executed to generate a display image including the ultrasound moving image output by the image processing function 142 and the time phase information output by the time phase identification function 144, and output the generated display image to the display device 106 so that the display device 106 is allowed to display the display image (step S108). More specifically, the display control function 146 is executed to arrange the ultrasound moving images output by the image processing function 142 within the image display area IA, display the time phase name included in the time phase information output by the time phase identification function 144 in the time phase display area PA, generate a display image showing the time information associated with the ultrasound moving image in the time axis area TA, and cause the display device 106 to display the display image. As a result, the ultrasound moving images captured up to the current time, as well as time information and time phase information about the ultrasound moving images, are sequentially presented to the examiner.

When the examiner specifies (selects) an ultrasound still image of one frame constituting the ultrasound moving image (step S110), the display control function 146 outputs information indicating the ultrasound still image of the one frame specified (selected) by the examiner to the image processing function 142. When the examiner does not specify (select) the ultrasound still image from the ultrasound moving image, the display control function 146 returns the process to step S104.

The image processing function 142 is executed to generate a new ultrasound still image from the ultrasound still images indicated in the information output by the display control function 146 (step S112). The image processing function 142 is executed to output the generated ultrasound still image to each of the time phase identification function 144 and the display control function 146.

The time phase identification function 144 is executed to identify the time phase of the ultrasound still image on the basis of the time information associated with the ultrasound still image output by the image processing function 142 (step S114). The time phase identification function 144 is executed to output the identified time phase information to the display control function 146.

The display control function 146 is executed to generate a display image obtained by replacing an ultrasound moving image specified (selected) by the examiner among the ultrasound moving images arranged within the image display area IA in the display image output to the display device 106 with an ultrasound still image output by the image processing function 142. The display control function 146 is executed to output the generated display image to the display device 106 so that the display device 106 is allowed to display the display image (step S116). Thereby, the specified (selected) ultrasound still image and the time information and time phase information related to this ultrasound still image are presented to the examiner.

When the examiner issues an instruction to provide examination information (step S120), the display control function 146 is executed to output (provide) the examination information based on the instruction to an information receiver (step S122). When the output of the examination information to the information receiver has been completed or when the examiner has not issued an instruction to provide the examination information, the display control function 146 is executed to return the process to step S104.

Subsequently, each constituent element provided in the medical image processing function 140 repeats each process. More specifically, the image processing function 142 is executed to repeat a process of generating an ultrasound still image based on the reflected wave signal output by the acquisition function 120, generating an ultrasound moving image by combining the ultrasound still images, and outputting the generated ultrasound moving image to the time phase identification function 144 and the display control function 146 in the processing of step S104 and a process of generating an ultrasound still image indicated in the information output by the display control function 146, and outputting the generated ultrasound still image to the time phase identification function 144 and the display control function 146 in the processing of step S112. The time phase identification function 144 is executed to repeat a process of identifying a time phase based on time information associated with the ultrasound moving image and outputting the identified time phase information to the display control function 146 in the processing of step S106 and a process of identifying a time phase based on time information associated with the ultrasound still image and outputting the identified time phase information to the display control function 146 in the processing of step S114. The display control function 146 is executed to repeat a process of generating a display image and outputting the generated display image to the display device 106 in the processing of step S108 and a process of generating a display image by replacing the ultrasound moving image arranged in the image display area IA with an ultrasound still image and outputting the generated display image to the display device 106 in the processing of step S116. Thereby, the ultrasound moving images captured up to the current time point and the time information and time phase information related to the specified ultrasound still image are sequentially presented to the examiner.

When the examiner ends the ultrasound contrast examination using the ultrasound diagnostic device 1, the acquisition function 120 is executed to end a process of acquiring the reflected wave signal output by the ultrasound probe 102 and outputting the acquired reflected wave signal to the medical image processing function 140 and the time measurement function 130 is executed to end a process of outputting the measured time information to the medical image processing function 140 by stopping the time measurement.

With such a configuration and process, in the ultrasound diagnostic device 1, the medical image processing function 140 in the processing circuitry 110 of the main body device 100 is executed to generate ultrasound still images on the basis of the reflected wave signals output by the acquisition function 120 and generate an ultrasound moving image in which ultrasound still images for a preset (programmed) period are combined. Furthermore, the medical image processing function 140 is executed to identify the time phase in the ultrasound contrast examination on the basis of the time information associated with the generated ultrasound moving image. Also, the medical image processing function 140 is executed to generate display images (display screens) in which the generated ultrasound moving image, the time information associated with the ultrasound moving image, and the identified time phase information are associated and sequentially output the generated display images to the display device 106 so that the display device 106 is allowed to display the display images. Thereby, the examiner can easily ascertain the image capturing situation of the ultrasound moving image in the ultrasound contrast examination and the display situation of the captured ultrasound moving image on the basis of the display image displayed on the display device 106. Also, the examiner can ascertain the ultrasound moving image and easily specify an ultrasound still image that is important in each time phase of the ultrasound contrast examination from the ultrasound moving image from among a plurality of ultrasound still images included in the ultrasound moving image.

When an ultrasound still image is specified from the ultrasound moving image by the examiner, the medical image processing function 140 within the processing circuitry 110 of the main body device 100 is executed to extract (pull out) the specified ultrasound still image from the ultrasound moving image and generate it as a new ultrasound still image in the ultrasound diagnostic device 1. Furthermore, the medical image processing function 140 is executed to identify the time phase in the ultrasound contrast examination on the basis of the time information associated with the generated ultrasound still image. Also, the medical image processing function 140 is executed to replace the original ultrasound moving image with the generated ultrasound still image, generate display images (display screens) in which the time information associated with the ultrasound still image is associated with the identified time phase information, and sequentially output the generated display images to the display device 106 so that the display device 106 is allowed to display the display images. Thereby, the examiner can obtain a diagnostic opinion of the ultrasound contrast examination on the basis of the display images displayed on the display device 106.

Furthermore, in the ultrasound diagnostic device 1, the medical image processing function 140 within the processing circuitry 110 provided in the main body device 100 is executed to provide various information related to the ultrasound contrast examination included in the display image to an information receiver, such as an application to be used by the examiner when a diagnostic opinion is input and a report is created. Thereby, the examiner can accurately link the specified ultrasound still image, the time information associated with this ultrasound still image, and the diagnostic opinion, and efficiently summarize the results of the ultrasound contrast examination. As described above, in the ultrasound diagnostic device to which the medical image processing device of the embodiment is applied, a display image including an ultrasound image (an ultrasound still image or an ultrasound moving image) based on the reflected wave signal output by the ultrasound probe and various information related to the ultrasound contrast examination, such as time information and time phase information when this ultrasound image was captured, is generated and displayed on the display device. Thereby, an examiner who performs an ultrasound contrast examination using an ultrasound diagnostic device to which the medical image processing device of the embodiment is applied can easily specify important ultrasound images (ultrasound still images) in an ultrasound contrast examination by ascertaining a display image (a display screen) displayed on a display device, and can obtain a diagnostic opinion based on the specified ultrasound images. Furthermore, in an ultrasound diagnostic device to which the medical image processing device of the embodiment is applied, an ultrasound image (an ultrasound still image) included in a display image displayed on the display device and various information (examination information) related to an ultrasound contrast examination such as time information and time phase information displayed on the ultrasound image are provided to a receiver indicated by the examiner. Thereby, the examiner who performs the ultrasound contrast examination using an ultrasound diagnostic device to which the medical image processing device of the embodiment is applied can efficiently summarize the results of the ultrasound contrast examination.

In the above-described embodiment, the case where the medical image processing device of the embodiment generates an ultrasound moving image in which ultrasound still images of a preset (programmed) period are combined has been described. However, a period for which ultrasound still images are combined as an ultrasound moving image is not limited to a period programmed in advance, and may be indicated by the examiner during the ultrasound contrast examination. For example, the examiner may indicate start and end timings of the ultrasound moving image each time by performing an input operation on the input device at the timings of starting and ending the recording of the ultrasound moving image. In this case, the medical image processing device of the embodiment generates an ultrasound moving image by combining ultrasound still images of a period of the indicated start and end timings of the ultrasound moving image. It is only necessary for the operation and process of the medical image processing device in this case to be equivalent to the operation and process of the medical image processing device of the above-described embodiment. Therefore, detailed descriptions of the operation and process of the medical image processing device when an ultrasound moving image is generated for a period indicated by the examiner each time will be omitted.

Although an example in which the medical image processing device of the embodiment is applied to the ultrasound diagnostic device of the embodiment has been described in the above-described embodiment, this is merely one example. The medical image processing device of the embodiment may be implemented as an independent device by a computer device such as a personal computer or a server device incorporated in a network or a cloud computing system. In this case, the medical image processing device implemented as an independent device can present the ultrasound moving image or ultrasound still image, time information and time phase information in association with one another to the examiner like the ultrasound diagnostic device of the above-described embodiment by acquiring an ultrasound moving image associated with the elapsed time from a reference time (when a contrast agent is injected into the subject) as time information. The ultrasound moving image associated with the time information acquired at this time, for example, may be information stored in a system including other devices or equipment different from the medical image processing device implemented as an independent device, such as an ultrasound diagnostic device or a database system such as a medical image management system. In this case, it is only necessary for the functional configuration, operation, and process of the medical image processing device implemented as an independent device to be equivalent to the functional configuration, operation, and process of the medical image processing device of the embodiment applied to the ultrasound diagnostic device of the above-described embodiment.

Therefore, detailed descriptions of a functional configuration, operation, and process when the medical image processing device is implemented as an independent device are omitted.

The information provision function 148 may store, into a memory, various information (examination information) obtained by starting various applications (such as opinion input application, report creation application, and analysis application) in association with an ultrasound image (such as an ultrasound still image or an ultrasound moving image) at each time phase displayed on the display screen DI of the display device 106. For example, when the opinion input application or the report creation application conforms to Liver Imaging Reporting and Data System (LI-RADS), the examiner uses the input device 104 to input an opinion on the ultrasound image (such as an ultrasound still image or an ultrasound moving image) at each time phase and a category (such as LR-1 (Definitely benign), LR-2 (Probably benign), LR-3 (Intermediate probability), LR-4 (Probably HCC), or LR-5 (Definitely HCC)) defined in LI-RADS. These categories are indicators indicating the condition or progress of lesion. The examiner performs an operation of selecting one category from among the categories. The information provision function 148 may store, into the memory, the category input by the examiner on an opinion input screen or report creation screen in association with an ultrasound image (such as an ultrasound still image or an ultrasound moving image) at each time phase displayed on the display screen DI of the display device 106.

Furthermore, when the analysis application conforms to LI-RADS, the analysis application may analyze the ultrasound image (such as an ultrasound still image or an ultrasound moving image) at each time phase to output a category defined in LI-RADS as examination information. In this case, the information provision function 148 may store, into the memory, the category obtained by starting the analysis application on the opinion input screen or report creation screen in association with an ultrasound image (such as an ultrasound still image or an ultrasound moving image) displayed on the display screen DI of the display device 106.

Furthermore, the information provision function 148 may store, into the memory, various information (examination information) obtained by starting various applications (such as opinion input application, report creation application, and analysis application) in association with an ultrasound image (such as an ultrasound still image or an ultrasound moving image) at each time phase stored in that memory. That is, images associated with the examination information may include an image in the captured image non-display period TSN shown in FIG. 5 (a) as well as the ultrasound image (such as an ultrasound still image or an ultrasound moving image) at each time phase displayed on the display screen DI of the display device 106.

Furthermore, the information provision function 148 may store, into the memory, various information (examination information) obtained by starting various applications (such as opinion input application, report creation application, and analysis application) in association with an ultrasound image (such as an ultrasound still image or an ultrasound moving image) re-stored in that memory. That is, images associated with the examination information may include an image with the still image restore mark RS shown in FIG. 5 (b) as well as the ultrasound image (such as an ultrasound still image or an ultrasound moving image) at each time phase displayed on the display screen DI of the display device 106.

The above-described embodiment can be represented as follows.

A medical image processing device including:
processing circuitry,
wherein the processing circuitry
sequentially generates ultrasound still images associated with time information indicating an elapsed time until a reflected wave signal is collected from a reference time on the basis of a reflected wave signal when an ultrasound signal transmitted toward the subject is reflected inside a body of the subject;
generates an ultrasound moving image including the ultrasound still images for a period according to a predetermined image capturing situation for a purpose of an ultrasound examination;
identifies the image capturing situation on the basis of the time information associated with the ultrasound still images included in the ultrasound moving image;
outputs identification information indicating the identified image capturing situation; and
causes a display device to display the ultrasound image corresponding to the identification information that has been selected and the time information when an ultrasound image of at least one of the ultrasound moving image and the ultrasound still images, the time information, and the identification information are displayed in association with one another.

According to at least one embodiment described above, there is provided processing circuitry configured to sequentially generate ultrasound still images associated with time information indicating an elapsed time until a reflected wave signal is collected from a reference time (when a contrast agent is injected into a subject) on the basis of a reflected wave signal when an ultrasound signal transmitted toward the subject is reflected inside a body of the subject; generate an ultrasound moving image including the ultrasound still images for a period according to a predetermined image capturing situation (time phase) for a purpose of an ultrasound examination (an ultrasound contrast examination) (142); identify the image capturing situation on the basis of the time information associated with the ultrasound still images included in the ultrasound moving image; output identification information indicating the identified image capturing situation (a time phase name indicating the time phase that has been identified or the like) (144); and cause a display device (106) to display an ultrasound image of at least one of the ultrasound moving image and the ultrasound still images, the time information, and the identification information in association with one another (146), wherein the processing circuitry causes the display device to display a display image (a display screen) including the ultrasound image corresponding to the identification information that has been selected and the time information by generating the display image (the display screen), whereby the ultrasound image captured in the ultrasound examination (the ultrasound contrast examination) can be presented to an examiner together with information about the image capturing situation of the ultrasound image and the ultrasound examination.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing device comprising:
processing circuitry configured to
sequentially generate ultrasound still images associated with time information indicating an elapsed time until a reflected wave signal is collected from a reference time based on a reflected wave signal when an ultrasound signal transmitted toward a subject is reflected inside a body of the subject;
generate an ultrasound moving image including the ultrasound still images for a period according to a predetermined image capturing situation for a purpose of an ultrasound examination;
identify the image capturing situation based on the time information associated with the ultrasound still images included in the ultrasound moving image;
output identification information indicating the identified image capturing situation; and
cause a display device to display an ultrasound image of at least one of the ultrasound moving image and the ultrasound still images, the time information, and the identification information in association with one another,
wherein the processing circuitry causes the display device to display the ultrasound image corresponding to the identification information that has been selected and the time information.

2. The medical image processing device according to claim 1,
wherein the processing circuitry is configured to:
cause the display device to display an identification information area in which the identification information is arranged and displayed in chronological order in which ultrasound images were captured, and
wherein the identification information area includes an identification information selection area for selecting the identification information.

3. The medical image processing device according to claim 2,
wherein the processing circuitry is configured to:
cause the display device to display an image display area in which a plurality of ultrasound images selected in the identification information selection area are arranged and displayed in chronological order in which the ultrasound images were captured.

4. The medical image processing device according to claim 3,
wherein the processing circuitry is configured to:
cause the display device to display a time axis area in which a total examination time from the reference time in the ultrasound examination is represented on a time axis and in which at least one of an imaging period of the ultrasound moving image indicated in the time information and times at which the ultrasound images were captured are represented on the time axis.

5. The medical image processing device according to claim 4,
wherein the processing circuitry is configured to:
display different time display aspects shown in the time axis area so that the ultrasound image displayed in the image display area and the ultrasound image not displayed in the image display area are distinguished.

6. The medical image processing device according to claim 5,
wherein, when any one ultrasound image included in the ultrasound moving image is specified in the ultrasound moving image displayed in the image display area,
wherein the processing circuitry is configured to:
output the identification information for identifying the image capturing situation based on the time information associated with the specified ultrasound image;
add a sub-number to the identification information of the specified ultrasound image;
display the identification information with the sub-number and the identification information selection area in the identification information area;
replace the ultrasound moving image displayed in the image display area with the specified ultrasound image; and
make a change from a time display aspect corresponding to the ultrasound moving image displayed in the time axis area to a time display aspect corresponding to the specified ultrasound image.

7. The medical image processing device according to claim 6,
wherein the processing circuitry is configured to:
provide the ultrasound still image displayed in the image display area, the time information of the ultrasound still image, and the identification information of the ultrasound still image as examination information in the ultrasound examination.

8. The medical image processing device according to claim 1,
wherein the reference time is a timing at which a contrast agent is injected into the subject when the ultrasound examination is performed.

9. The medical image processing device according to claim 8,
wherein the image capturing situation is a time phase indicating a situation of an examination target area within the body of the subject determined by the elapsed time from the reference time, and
wherein the identification information is a time phase name indicating the time phase that has been identified.

10. An ultrasound diagnostic device comprising:
an ultrasound probe configured to transmit an ultrasound signal to a subject, detect a reflected wave signal when the ultrasound signal is reflected inside a body of the subject, and output the detected reflected wave signal; and
a medical image processing device including processing circuitry configured to
acquire the reflected wave signal output by the ultrasound probe;
start time measurement in accordance with an instruction to output time information indicating a measured time;
sequentially generate ultrasound still images associated with time information indicating an elapsed time until a reflected wave signal is collected from a reference time based on the reflected wave signal
generate an ultrasound moving image including the ultrasound still images for a period according to a predetermined image capturing situation for a purpose of an ultrasound examination;
identify the image capturing situation based on the time information associated with the ultrasound still images included in the ultrasound moving image;
output identification information indicating the identified image capturing situation; and
cause a display device to display an ultrasound image of at least one of the ultrasound moving image and the ultrasound still images, the time information, and the identification information in association with one another,
wherein the processing circuitry is configured to:
cause the display device to display the ultrasound image corresponding to the identification information that has been selected and the time information.

11. A medical image processing method comprising:
sequentially generating, by a computer, ultrasound still images associated with time information indicating an elapsed time until a reflected wave signal is collected from a reference time based on a reflected wave signal when an ultrasound signal transmitted toward a subject is reflected inside a body of the subject;
generating, by the computer, an ultrasound moving image including the ultrasound still images for a period according to a predetermined image capturing situation for a purpose of an ultrasound examination;
identifying, by the computer, the image capturing situation based on the time information associated with the ultrasound still images included in the ultrasound moving image;
outputting, by the computer, identification information indicating the identified image capturing situation; and
causing, by the computer, a display device to display the ultrasound image corresponding to the identification information that has been selected and the time information when an ultrasound image of at least one of the ultrasound moving image and the ultrasound still images, the time information, and the identification information are displayed in association with one another.

12. A non-transitory computer-readable storage medium storing a program for causing a computer to
sequentially generate ultrasound still images associated with time information indicating an elapsed time until a reflected wave signal is collected from a reference time based on a reflected wave signal when an ultrasound signal transmitted toward a subject is reflected inside a body of the subject;
generate an ultrasound moving image including the ultrasound still images for a period according to a predetermined image capturing situation for a purpose of an ultrasound examination;
identify the image capturing situation based on the time information associated with the ultrasound still images included in the ultrasound moving image;
output identification information indicating the identified image capturing situation; and
cause a display device to display the ultrasound image corresponding to the identification information that has been selected and the time information when an ultrasound image of at least one of the ultrasound moving image and the ultrasound still images, the time information, and the identification information are displayed in association with one another.
